(19) United States
(12) Patent Application Publication
(10) Pub. No.: US 2012/0316489 A1
(43) Pub. Date: Dec. 13, 2012

(54) METHOD AND SYSTEM FOR TREATING CANCER

(76) Inventor: Der-Yang TIEN, Pasadena, CA (US)

(21) Appl. No.: 13/592,839

(22) Filed: Aug. 23, 2012

Related U.S. Application Data
(63) Continuation-in-part of application No. 12/888,245, filed on Sep. 22, 2010.
Application No. 12/888,245 is a continuation-in-part of application No. 12/194,497, filed on Aug. 19, 2008.
Application No. 12/194,497 is a continuation-in-part of application No. 12/121,712, filed on May 15, 2008.
Application No. 12/121,712 is a continuation of application No. 10/767,387, filed on Jan. 28, 2004, now Pat. No. 7,415,302.

(30) Foreign Application Priority Data
Oct. 15, 2003 (TW) 092128522

(51) Int. Cl. A61M 37/00

(52) U.S. Cl. 604/21

(57) ABSTRACT

A method and system for treating cancer of a patient are disclosed. In a first aspect, the method comprises injecting bleomycin into the patient and administering excorporeal ultrasound to a localized area of the cancer, wherein the bleomycin more readily enters the localized area to treat the cancer of the patient. In a second aspect, the system comprises an injection unit for injecting bleomycin into the patient and an ultrasound module for administering excorporeal ultrasound to a localized area of the cancer, wherein the bleomycin more readily enters the localized area to treat the cancer of the patient.

### CROSS-REFERENCE TO RELATED APPLICATIONS

This application is a continuation-in-part of U.S. patent application Ser. No. 12/888,245, filed Sep. 22, 2010, entitled “METHOD AND SYSTEM FOR LEADING MACROMOLECULE SUBSTANCES INTO LIVING TARGET CELLS,” which is a continuation-in-part of U.S. patent application Ser. No. 12/194,497, filed Aug. 19, 2008, entitled “METHOD AND SYSTEM FOR LEADING MACROMOLECULE SUBSTANCE INTO LIVING TARGET CELLS,” which is a continuation-in-part of U.S. patent application Ser. No. 12/121,712, filed May 15, 2008, entitled “METHOD AND SYSTEM FOR LEADING MACROMOLECULE SUBSTANCES INTO LIVING TARGET CELLS,” which is a continuation of U.S. Pat. No. 7,415,302, issued Aug. 19, 2008, entitled “METHOD AND SYSTEM FOR LEADING MACROMOLECULE SUBSTANCES INTO LIVING TARGET CELLS,” which claims priority to Taiwan Patent Application No. 092128522, filed Oct. 15, 2003, all of which are incorporated herein by reference.

### FIELD OF THE INVENTION

The present invention relates generally to treating cancer, and more particularly, to a method and system for treating cancer using pain management and cancer growth inhibition techniques.

### BACKGROUND

At the present time, approximately 5,000,000 people die every year because of cancer, and malignant tumors are the main reason. With the development of medical sciences, cancer diagnostic and treatment methods exist such as surgery, chemotherapy, and actinotherapy. Achieving maximum curative effect both in the treatment of the cancer and the related pain management while achieving minimum destruction of healthy cells and low medicine dosages are important therapeutic goals.

Pain management for patients with cancer is one of the most important aspects of their care. Pain is the most common presenting symptom in patients with various forms of cancer and should be controlled. The best management of pain is aggressive therapy with constant assessment. The patient with cancer who is experiencing pain can maintain his/her quality of life with adequate pain management.

Current pain management treatments include using opioids that carry obvious side effects and invasive surgical techniques of pain control. There is a strong need for a cost-effective and non-invasive cancer treatment and pain management process to treat various forms of cancer, including but not limited to pancreatic cancer, in order to keep a quality life in cancer patients. The present invention addresses such a need.

### SUMMARY OF THE INVENTION

A method and system for treating cancer of a patient are disclosed. In a first aspect, the method comprises injecting bleomycin into the patient and administering excorporeal ultrasound to a localized area of the cancer, wherein the bleomycin more readily enters the localized area to treat the cancer of the patient. In a second aspect, the system comprises an injection unit for injecting bleomycin into the patient and an ultrasound module for administering excorporeal ultrasound to a localized area of the cancer, wherein the bleomycin more readily enters the localized area to treat the cancer of the patient.

### BRIEF DESCRIPTION OF THE DRAWINGS

FIG. 1A illustrates a basic structure of a system for leading macromolecule substances into living target cells in accordance with an embodiment.

FIGS. 1B-1D are a perspective, front, and side view respectively, of an ultrasound wave energy conversion module utilized for energy conversion in accordance with an embodiment.

FIG. 1E illustrates several low energy ultrasound transducers or tweeters 150 located around the peripheral of the disk of the ultrasound dispersion unit and symmetrically positioned in accordance with an embodiment.

FIG. 1F illustrates that when the symmetrically positioned low energy ultrasound transducers or tweeters are within a merge zone of 20 cm from the disk, the ultrasound intensity at the merge zone is in a range of 0.2-0.3 W/cm².

FIG. 1G illustrates (on the left side) a 3D co-registration of tumor mass and its vessels and illustrates (on the right side) an injection of artificial blood perfluorocarbon nanoemulsion (tiny white dots) into the tumor vessels which fills the tumor interstitial space.

FIG. 1H illustrates (larger picture) a design of the imaging guided robotic arm of the ultrasound wave energy conversion module in accordance with an embodiment.

FIG. 1I illustrates that with computerized imaging guidance the focal zone of the ultrasound wave energy conversion module is precisely positioned in a desirable treatment area within the tumor mass with the assistance of the imaging guide robotic arm.

FIG. 1J illustrates a schematic demonstration of a tumor before and after treatment in accordance with an embodiment.

FIG. 1K illustrates that the imaging guided robotic arm can be an independent entity or can be coupled to an imaging device.

FIG. 2 illustrates a method for leading macromolecule substances into living target cells using the system 1 in accordance with an embodiment.

FIG. 3 illustrates a method for treating cancer of a patient in accordance with an embodiment.

FIG. 4 illustrates a system for treating cancer of a patient in accordance with an embodiment.

### DETAILED DESCRIPTION

The present invention relates generally to treating cancer, and more particularly, to a method and system for treating cancer using pain management and cancer growth inhibition techniques. The following description is presented to enable one of ordinary skill in the art to make and use the invention and is provided in the context of a patent application and its requirements. Various modifications to the preferred embodiment and the generic principles and features described herein will be readily apparent to those skilled in the art. Thus, the present invention is not intended to be limited to the embodiment shown but is to be accorded the widest scope consistent with the principles and features described herein.

Pain is the most common presenting symptom in patients with various forms of cancer, especially in patients with pancreatic cancer. For pancreatic cancer patients, the pain is typically mid epigastric in location, with radiation of the pain occurring to the mid or lower back region. Radiation of the pain to the back is worrisome, as it indicates retroperitoneal invasion of the splanchnic nerve plexus by the cancer. Often, the pain is unrelenting in nature with night-time pain often being a predominant complaint. Some patients note increased discomfort after eating and when the patient is lying flat. All patients experience pain at some point in their clinical course.

Pain in cancer patients is also causally linked to a prevalence of depressive disorders of all types. This link between pain and depression, along with anxiety and other mood disorders, underscores the problem of undertreatment for pain as the most common opioid abuse issue in the care of the cancer patients. Pain management is an aspect of cancer treatment that is most worrisome to both patients and their families. The paradox of cancer pain is the following: it is the most feared symptom, the most connected and interwoven symptom to other cancer symptoms (e.g. insomnia, fatigue, nausea, constipation, etc.), and yet treatable.

Pancreatic cancer pain syndromes occur due to the proximity of the organ to a number of other critical structures (e.g. the duodenum, liver, stomach, jejunum, transverse colon, etc.). The pancreas is innervated by nerve networks that interact with both the parasympathetic and sympathetic nervous systems so pancreatic cancer pain is felt at multiple and distant sites.

Most patients with chronic malignant pain require an opioid regime consisting of around-the-clock dosing, with a “rescue” dose calculated at approximately 15% of the 24-hour baseline dose. Oral doses can be given at every hour for relief, and the severity of the pain determines the dose, route, and frequency of the analgesic intervention. Finding the correct opioid dosage is empirical utilizing trial and error. Common reasons for inadequate pain control include making errors in dosing, failing to escalate total and breakthrough dose, not addressing side effects, and not using alternative opioids and adjuvant analgesics (e.g. antidepressants, anticonvulsants, corticosteroids, etc.). The final opioid dose require for pain relief is the dose that works with an acceptable side effect profile. Thus, the dosing requirements necessary to deliver adequate pain relief vary widely among patients.

Percutaneous celiac plexus blockage is an adjunctive interventional technique utilized in patients whose pain is poorly controlled with opioids and who are bothered by escalating adverse effects. In certain cases, the procedure can cause significant complications. Intraspinal drug delivery either intrathecal or epidural and implantable drug delivery systems can also treat select patients with intolerable pain. Radiotherapy is principally used as a palliative modality.

A method and system in accordance with the present invention provides for treating pancreatic cancer of a patient by utilizing intravenous delivery of bleomycin in conjunction with non-invasive excorporeal low frequency ultrasound to effectively deliver bleomycin into pancreatic cancer tissues of the patient. Bleomycin is very water soluble and thus penetrates the cell membrane with poor efficiency. Thus, the low frequency ultrasound assists the bleomycin delivery into the pancreatic cancer tissue. Although Bleomycin has been used to treat various cancers including lymphomas and head and neck cancers, it has not been used successfully as an anticancer agent to treat pancreatic cancer and has never been used as a pain management technique to treat any type of cancer.

Bleomycin for injection USP is a mixture of cytotoxic glycopeptides antibiotics isolated from a strain of Streptomyces verticillus. Bleomycin is freely soluble in water and is provided for injection as a sterile, white to off-white, lyophilized cake or powder in vials for intramuscular, intravenous, or subcutaneous administration. The chemical name is N1-[3-(dimethylsulphonio)propyl]Bleomycin-amide (Bleomycin A2) and is N1-4-(guanidobutyl)Bleomycinamide (Bleomycin B2). The molecular formula of Bleomycin A2 is C55H84N17021S3 with a calculated molecular weight of 1414 and the molecular formula of Bleomycin B2 is C55H84N20021S2 with a calculated molecular weight of 1425. Bleomycin causes single and double stranded breaks in DNA to inhibit DNA, RNA, and protein synthesis and to cause cell cycle arrest and apoptosis.

The main advantage of ultrasound is its non-invasive nature: the transducer of the ultrasound is placed in contact with a water-based gel on the skin of the patient, and no insertion or surgery is thus required. The interactions of ultrasound with biological tissues are divided into thermal and non-thermal effect categories. Accordingly, utilization of a low frequency and tailored ultrasound procedure is advantageous. Ultrasound causes chemical reactions that are chemotherapeutic and aide in the delivery of therapeutic drugs to vital organ and tissue areas.

To describe the features of the present invention in more detail, refer now to the following description in conjunction with the accompanying Figures.

One of ordinary skill in the art readily recognizes that a system for leading macromolecule substances, including but not limited to bleomycin, into living target cells can be applied to a variety of different fields, such as gene delivery, gene therapy, medicine transmission, partial medication, and tumor treatment and that would be within the spirit and scope of the present invention. In a solid tumor treatment, for example, a preparatory step is usually taken by computed tomography (CT) or magnetic resonance imaging (MRI). Three-dimensional (3D) structure images of the tissue or organ where tumor cells locate are picked up by the preparatory step, as a basis for subsequent treatments (such as surgery, chemotherapy and actinotheraphy).

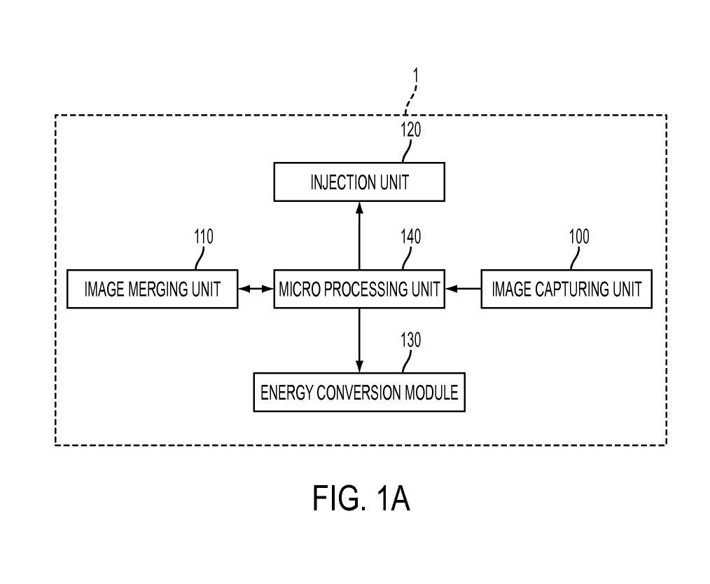

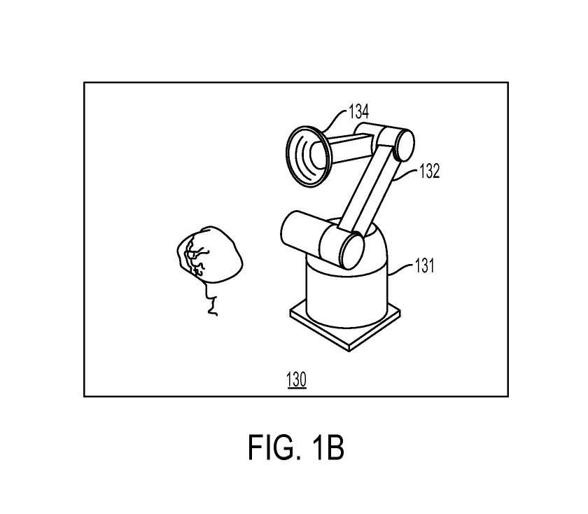

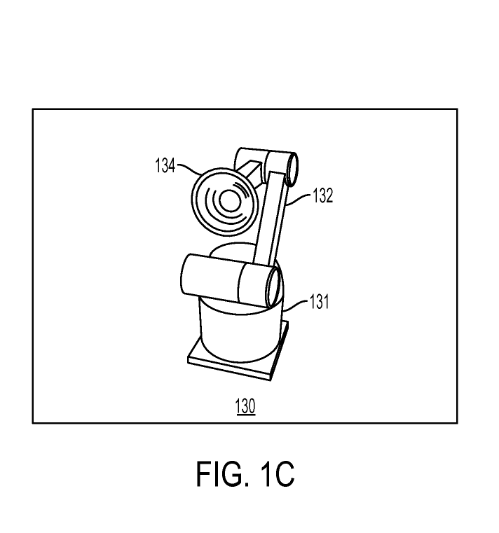

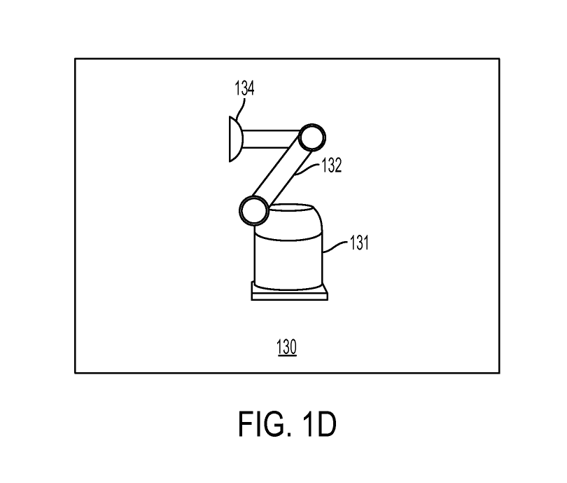

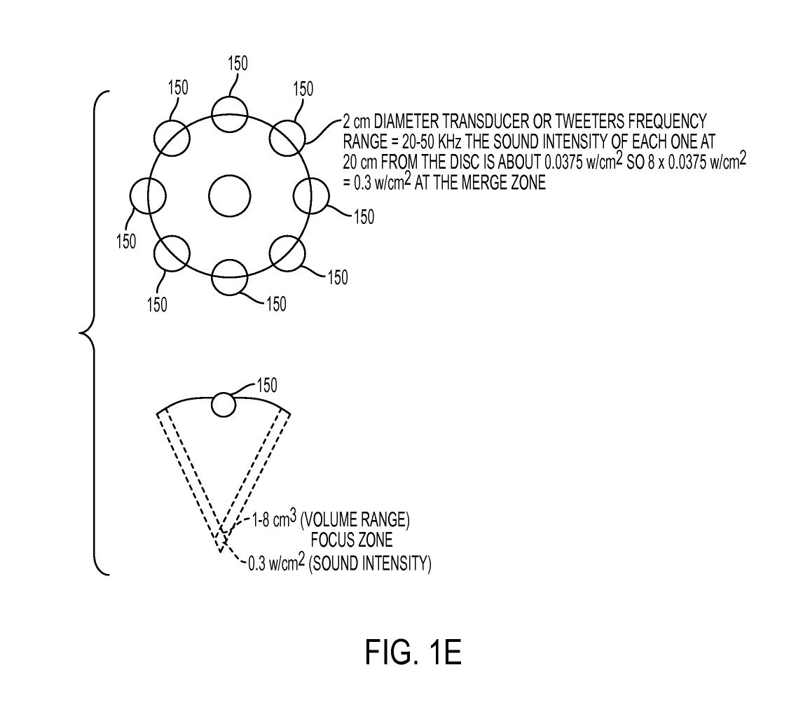

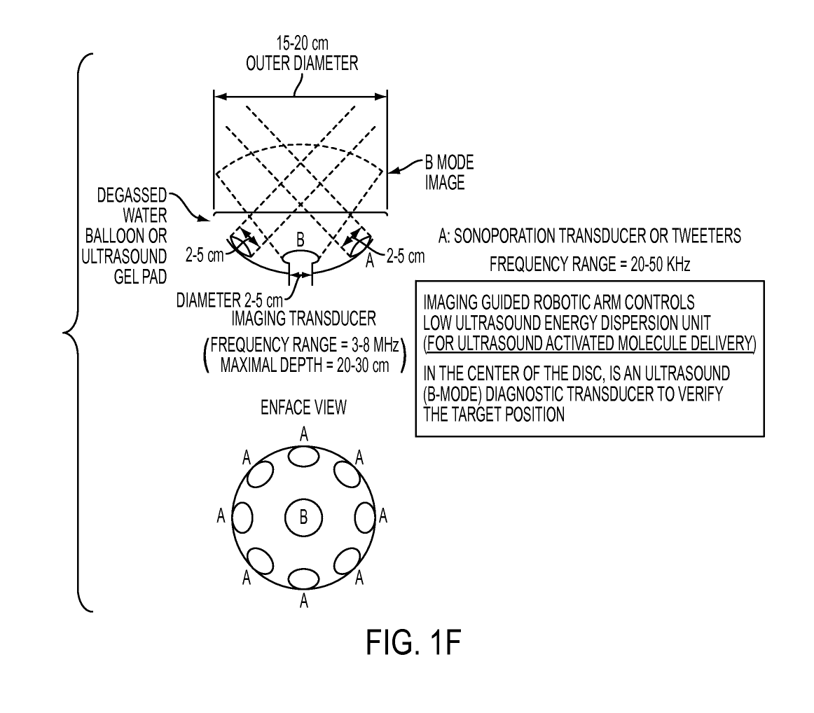

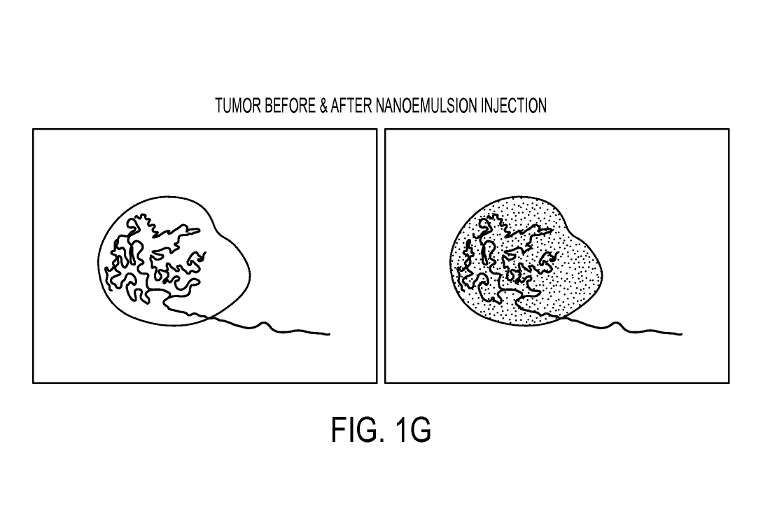

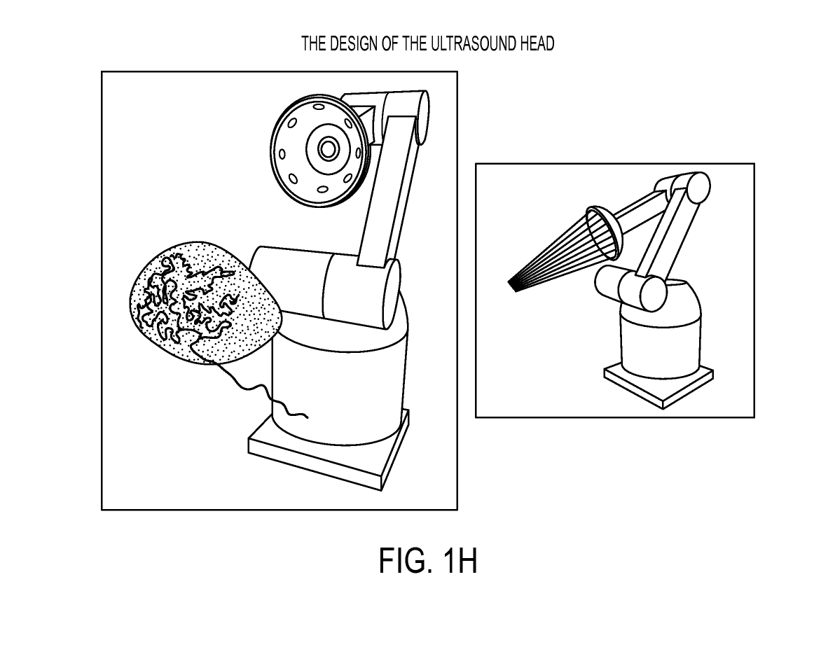

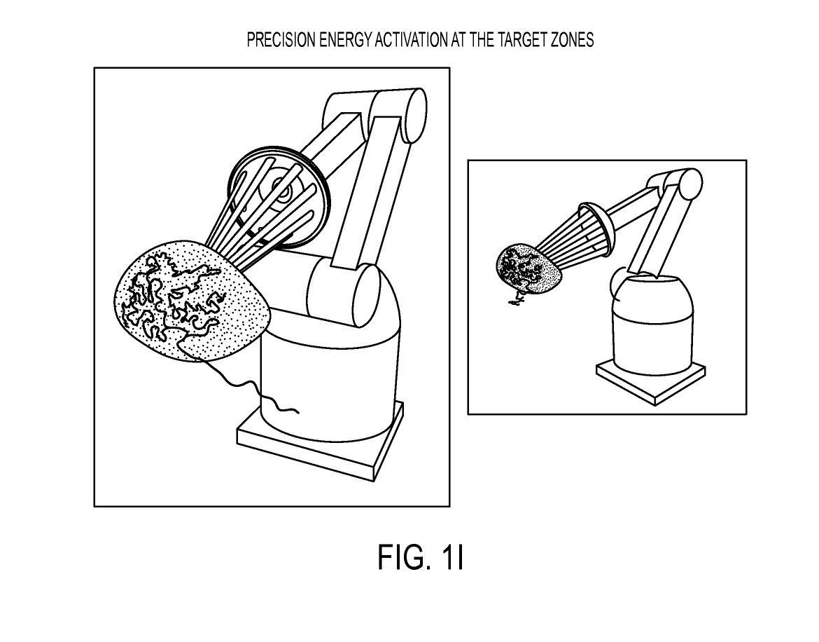

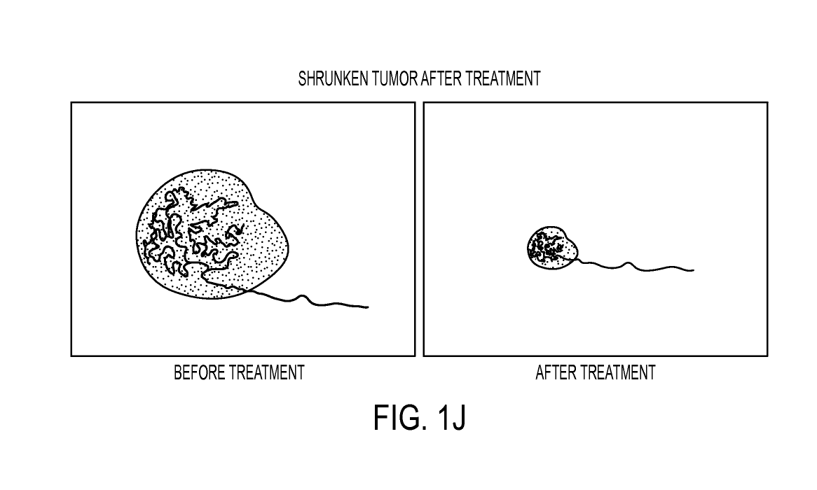

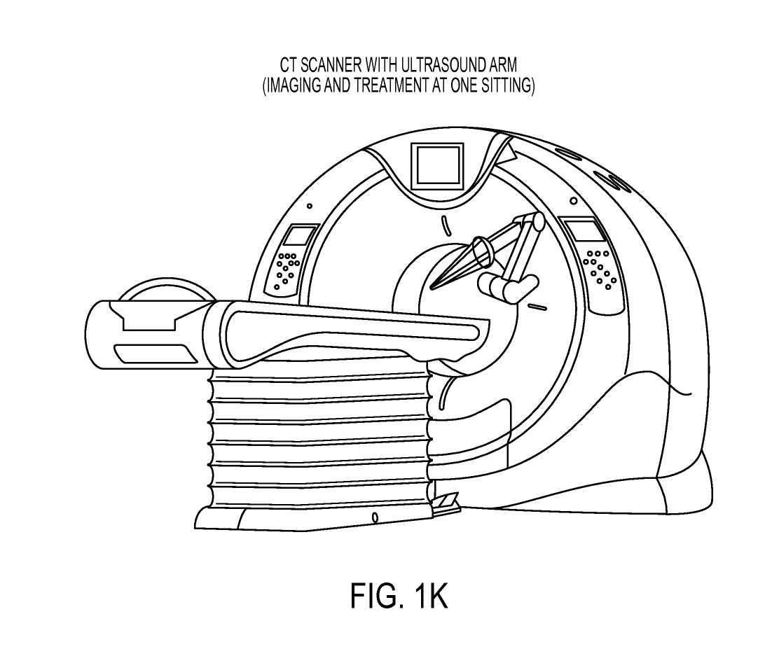

FIG. 1A illustrates a basic structure of a system 1 for leading macromolecule substances into living target cells in accordance with an embodiment. The system 1 includes an image picking unit 100, an image merging unit 110, an injection unit 120, an energy conversion module 130, and a micro processing unit 140 controlling the image picking unit 100, the image merging unit 110, the injection unit 120, and the energy conversion module 130. In one embodiment, the living target cells are cancer cells.

The image picking unit 100 is applied for picking up 3D structure images of the tissue or organ where the target cells locate, and for picking up 3D photographic images of the blood vessel where the target cells locate. In one embodiment, the image picking unit 100 is one of a CT device, a MRI device, and a blood vessel photographic device.

Proper selection methods for picking up 3D structure images of a tissue or organ focus on where the tumor locates and the personal situation of the patient. Although CT and MRI devices pick up 3D structure images of a tissue or organ, these devices do not adequate pick up medicine transmission passages. To overcome this problem, the image picking unit 100 further includes a blood vessel photographic device in addition to a CT and/or MRI device.

The blood vessel photographic device injects a special developer into the blood vessel to generate a series of blood vessel images. For example, in checking a heart blood vessel system, femoral is firstly pierced from inguen, a pipe is then put in and conversely transmitted into particular blood vessel. The special developer is then quickly injected into the blood vessel through the pipe, and consecutive snapshots are simultaneously taken. Thus, the blood flow situation of the organ where the blood vessel flows into, such as brain, heart, liver or kidney, can be obtained. In one embodiment, the 3D blood vessel photographic images where the tumor cells locate are obtained by using 3D reconstructed blood vessel photography including but not limited to a diagnostic and interventional angiography system (Advantx LCA+), a cardiovascular and angiography imaging system (Advantx LCV+), and a biplane neuroangiography system (Advantx LCN+).

The image merging unit 110 merges or tissue maps the 3D structure image picked up by the image picking unit 100 into 3D blood vessel photographic images to precisely locate the tumor cells and to choose a proper blood vessel passage fully covering the tumor cells. Medicine is injected by the injection unit 120 along the chosen blood vessel passage to ensure the medicine is efficiently transmitted to the tumor cells.

The injection unit 120 applies a pipe along the chosen blood vessel passage for injecting therapeutic substances into the tumor cells and surrounding tissue. In one embodiment, the therapeutic substances are any of any of a macromolecular substances, medicine, and bleomycin. The therapeutic substances enter into the tumor cells and surrounding tissue through non-permanent holes formed by a sonoporation process that utilizes tiny bubbles liquid. The size of the bubble is preferred to be smaller than 10 micron to enable smooth passing through the blood vessel.

The therapeutic substances can be injected before or after the sonoporation process. Because the therapeutic substances enter into the tumor cells through the non-permanent holes formed in the tumor cell membrane, the therapeutic substance dosage is reduced to approximately 1% of a normal dosage thereby achieving a more efficient curative effect with decreased costs and damage to healthy cells/tissue.

The energy conversion module 130 exerts energy to activate the medicine to perform biological effects to form non-permanent holes in the cell membranes of the tumor cells. In one embodiment, the energy conversion module 130 is an ultrasonic wave conversion module that includes ultrasonic transducers or tweeters that exert ultrasonic waves of 20-50 KHz frequency.

FIGS. 1B-1D are a perspective, front, and side view respectively, of an ultrasound wave energy conversion module 130 utilized for energy conversion in accordance with an embodiment. The ultrasound wave energy conversion module 130 includes a base portion 131, an imaging guided robotic arm 132, and an ultrasound dispersion unit 134. In one embodiment, the ultrasound dispersion unit 134 includes a disk with transducers or tweeters for radiating the ultrasound energy. The imaging guided robotic arm 132 controls the ultrasound dispersion unit 134 for ultrasound activated molecule delivery. In one embodiment, in the center of the disk is a ultrasound (B-Mode) diagnostic transducer to verify target positions.

FIG. 1E illustrates several low energy ultrasound transducers or tweeters 150 located around the peripheral of the disk of the ultrasound dispersion unit 134 and symmetrically positioned in accordance with an embodiment. In this embodiment, the frequency range of the low energy ultrasound transducers or tweeters 150 is 20-50 KHz and the energy merge zone adjustable intensity range is approximately 0.2-0.3 W/cm² (about 20 cm from the disk).

FIG. 1F illustrates that when the symmetrically positioned low energy ultrasound transducers or tweeters are within a merge zone of 20 cm from the disk, the ultrasound intensity at the merge zone is in a range of 0.2-0.3 W/cm². By using the ultrasound wave energy conversion module 130, efficient delivery of energy is provided to the targeted tumor cells.

FIG. 1G illustrates (on the left side) a 3D co-registration of tumor mass and its vessels and illustrates (on the right side) an injection of artificial blood perfluorocarbon nanoemulsion (tiny white dots) into the tumor vessels which fills the tumor interstitial space.

FIG. 1H illustrates (larger picture) a design of the imaging guided robotic arm 132 of the ultrasound wave energy conversion module 130 in accordance with an embodiment. The imaging guided robotic arm 132 includes a head disk with eight (8) symmetrically positioned low energy transducers or tweeters that have a frequency range of 20-50 KHz and a size of approximately 2 cm in diameter. The focal zone of these transducers or tweeters is about 20 cm from the disk surface. In this embodiment, the head disk is about 15-20 cm in diameter. There is a central positioned B-mode diagnostic transducer in the disk (frequency 3-8 MHz, diameter is 3-5 cm, maximal depth of penetration is 20-30 cm).

FIG. 1H also illustrates (smaller picture) that the focal zone (merge zone) of the peripheral transducers is located about 20 cm from the head disk. The focal zone's ultrasound energy level is approximately 0.2-0.3 W per square cm which is optimal for a low frequency ultrasound cavitation (sonoporation) effect and is well within FDA ultrasound safety guidelines. The paths of the eight individual ultrasound beams have very low ultrasound energy which can neither create sonoporation effects nor any undesirable physiological effects. Thus, only the focal zone has a therapeutic sonoporation effect that is also safe for patients.

FIG. 1I illustrates that with computerized imaging guidance the focal zone of the ultrasound wave energy conversion module 130 is precisely positioned in a desirable treatment area within the tumor mass with the assistance of the imaging guide robotic arm 132. FIG. 1J illustrates a schematic demonstration of a tumor before and after treatment. The tumor mass shrinks greatly after treatment. FIG. 1K illustrates that the imaging guided robotic arm 132 can be an independent entity or can be coupled to an imaging device (e.g. CT, MRI, PET scanners, etc.).

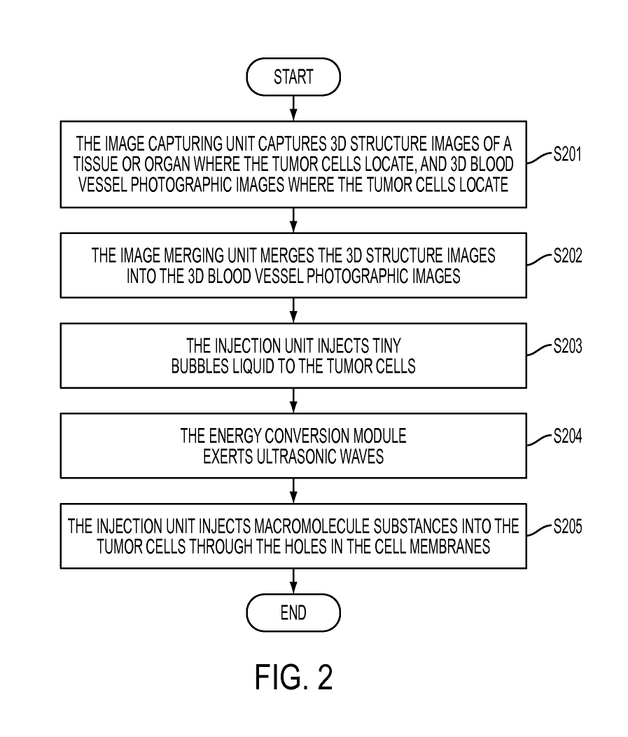

FIG. 2 illustrates a method for leading macromolecule substances into living target cells using the system 1 in accordance with an embodiment. In step S201, the image picking unit 100 picks up both 3D structure images of the tissue or organ where the tumor cells locate and 3D blood vessel photographic images where the tumor cells locate. In step S202, the image merging unit 110 merges the 3D structure images into the 3D blood vessel photographic images to precisely locate the tumor cells and to choose a blood vessel passage that fully covers the tumor cells.

In step S203, the injection unit 120 injects tiny bubbles liquid around the tumor cells through the chosen blood vessel passage. In step S204, the energy conversion module 130 exerts ultrasonic waves using the peripheral transducers or tweeters to activate the tiny bubbles liquid to perform biological effects thereby forming non-permanent holes in the cell membranes of the tumor cells. In step S205, the injection unit 120 injects macromolecule substances, including but not limited to medicine, through the non-permanent holes in the cell membranes of the tumor cells.

In one embodiment, artificial blood is injected around the tumor cells as the tiny bubbles liquid. The artificial blood has a small volume of approximately 150 nanometers, so that the capillary vessels are not jammed and so that the artificial blood does not enter into the apertures between the blood vessels. Thus, oxygen deficiency resulting from low blood flow when using an injection pipe is improved. In another embodiment, the artificial blood is perfluorocarbon (PFC) nanoemulsion.

In one embodiment, an ultrasonic wave developer is injected either by mainline or by using a pipe to pick up the 3D blood vessel photographic images. The ultrasonic wave developer is composed of tiny bubbles enwrapped in special protection housing. The tiny bubbles can enwrap air or various types of gas including but not limited to fluro-carbon. The size of the ultrasonic wave developer is approximately 10 micron so that it can smoothly pass through micro blood vessels.

When exerted with ultrasonic waves of 1 Mpa intensity, the bubbles of the ultrasonic wave developer perform non-linear oscillation, and emit harmonic signals. These harmonic signals are stronger and distinct from tissue signals which aids in the determination of the blood flow situations of various organs, tissues, and vessels.

In one embodiment, after medicine, including but not limited to bleomycin, is injected around the tumor cells, an application of ultrasonic waves of at least 1 Mpa intensity or shock waves of proper intensity using transducers or tweeters activate the tiny bubbles liquid or the ultrasonic wave developer to perform strong bubble movements which form non-permanent holes in the cell membranes of the tumor cells. Accordingly, the permeability of the cell membranes is increased enabling the medicine to more readily target the tumor cells while sharply lowering required medication dosage levels. In another embodiment, the medicine is injected before the application of the ultrasonic waves.

In one embodiment, the system 1 cooperates with a data processing electronic device to process data generated during the course of the operation of the system 1. One of ordinary skill in the art readily recognizes that the data processing electronic device can be a variety of devices including but not limited to a personal computer (PC), a notebook computer (NB), a server, a working station, a personal digital assistant (PDA), a Liquid Crystal Display (LCD) computer, and a tablet PC and that would be within the spirit and scope of the present invention.

In this embodiment, the data processing electronic device includes a display unit and an input unit. The display unit is used for displaying the images merging process performed by the image merging unit 110, the medicine injection process performed by the injection unit 120, and the energy transmitting process of the energy conversion module 130. The input unit is used for inputting commands and/or parameters of the system 1 to the data processing electronic device.

In one embodiment, as opposed to a localized injection, the bleomycin is intravenously injected into the bloodstream of a patient with pancreatic cancer so that the bleomycin is systemically circulated over the patient's entire body. After the intravenous injection, an ultrasound system is utilized to administer the non-invasive excorporeal low frequency ultrasound locally near the pancreas of the patient. The ultrasound system includes a power amplifier and at least two transducers coupled to the power amplifier. In one embodiment, a contact surface of the at least two transducers is aluminum. The power amplifier can be set to variable output levels and the AC input of the power amplifier requires 230 VAC.

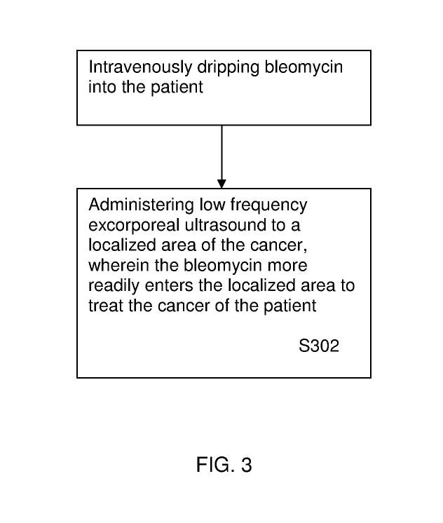

FIG. 3 illustrates a method 300 for treating cancer of a patient. In step S301, bleomycin is intravenously dripped into the patient. In step S302, low frequency excorporeal ultrasound is administered to a localized area of the cancer, wherein the bleomycin more readily enters the localized area to treat the cancer of the patient.

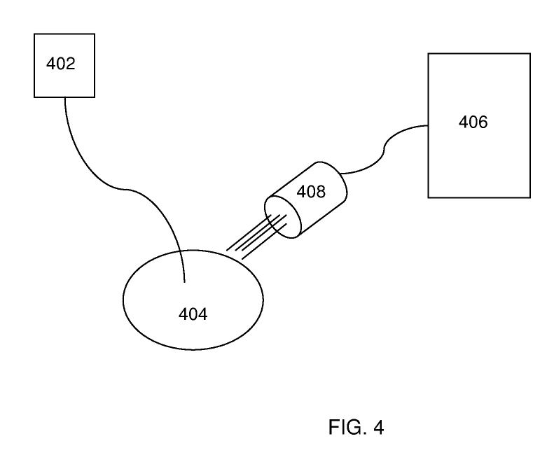

FIG. 4 illustrates a system 400 for treating cancer of a patient. The system 400 includes an intravenous drip unit 402 and a low frequency ultrasound unit 406 with at least one transducer 408. The intravenous drip unit 402 drips bleomycin into the patient 404 for a predetermined time period. Then, the low frequency ultrasound unit 406 administers ultrasound, via the at least one transducer 408, to a localized area of the patient where the cancer resides to enable the bleomycin to more readily enter into the localized area and manage pain of the patient.

As above described, the method and system in accordance with the present invention allow for tumor cell treatment and cancer related pain management by delivering bleomycin intravenously in conjunction with non-invasive excorporeal low frequency ultrasound. The ultrasound enables the bleomycin to more readily enter into the tumor cells and surrounding tissue and nerve fibers.

Although the present invention has been described in accordance with the embodiments shown, one of ordinary skill in the art will readily recognize that there could be variations to the embodiments and those variations would be within the spirit and scope of the present invention. Accordingly, many modifications may be made by one of ordinary skill in the art without departing from the spirit and scope of the appended claims.

What is claimed is:

1. A method for treating cancer of a patient, the method comprising:
injecting bleomycin into the patient; and
administering excorporeal ultrasound to a localized area of the cancer, wherein the bleomycin more readily enters the localized area to treat the cancer of the patient.

2. The method of claim 1, wherein the injecting comprises intravenously dripping bleomycin into a bloodstream of the patient.

3. The method of claim 1, wherein the cancer is pancreatic cancer.

4. The method of claim 1, wherein treating the cancer of the patient includes any of pain management treatment and cancer growth inhibition treatment.

5. The method of claim 1, wherein the excorporeal ultrasound uses low frequency at 27 KHz with low power intensity of 1 watt per square cm.

6. The method of claim 2, wherein the bleomycin is intravenously dripped into the bloodstream of the patient for a five to ten minute predetermined time period.

7. The method of claim 3, wherein the bleomycin treats the cancer of the patient by entering, through sonoporation, into nerves and ganglia of surrounding tissues and tissues of the pancreatic cancer to paralyze the tissues.

8. The method of claim 1, wherein the excorporeal ultrasound is a vibrating wave which oscillates target pancreatic cancer tissues and decreases ductal and interstitial pancreatic cancer tissue pressures to improve local circulation and to minimize mechanical stimuli to nerves connected to the pancreatic cancer tissue.

9. The method of claim 1, wherein the excorporeal ultrasound is administered by an ultrasound module that includes a base portion, an imaging guided robotic arm coupled to the base portion, and an ultrasound dispersion unit coupled to the imaging guide robotic arm, wherein the ultrasound dispersion unit comprises a disk with eight symmetrically positioned low energy transducers located on a peripheral of the disk.

10. The method of claim 9, wherein in a center of the disk is an ultrasound (B-Mode) diagnostic transducer to verify target positions.

11. A system for treating cancer of a patient, the system comprising:
an injection unit for injecting bleomycin into the patient; and
an ultrasound module for administering excorporeal ultrasound to a localized area of the cancer, wherein the bleomycin more readily enters the localized area to treat the cancer of the patient.

12. The system of claim 11, wherein the injection unit intravenously drips bleomycin into a bloodstream of the patient.

13. The system of claim 11, wherein the cancer is pancreatic cancer.

14. The system of claim 11, wherein treating the cancer of the patient includes any of pain management treatment and cancer growth inhibition treatment.

15. The system of claim 11, wherein the ultrasound module uses low frequency at 27 KHz with low power intensity of 1 watt per square cm.

16. The system of claim 12, wherein the bleomycin is intravenously dripped into the bloodstream of the patient for a five to ten minute predetermined time period.

17. The system of claim 13, wherein the bleomycin treats the cancer of the patient by entering, through sonoporation, into nerves and ganglia of surrounding tissues and tissues of the pancreatic cancer to paralyze the tissues.

18. The system of claim 11, wherein the excorporeal ultrasound is a vibrating wave which oscillates target pancreatic cancer tissues and decreases ductal and interstitial pancreatic cancer tissue pressures to improve local circulation and to minimize mechanical stimuli to nerves connected to the pancreatic cancer tissue.

19. The system of claim 11, wherein the ultrasound module includes a base portion, an imaging guided robotic arm coupled to the base portion, and an ultrasound dispersion unit coupled to the imaging guide robotic arm, wherein the ultrasound dispersion unit comprises a disk with eight symmetrically positioned low energy transducers located on a peripheral of the disk.

20. The system of claim 19, wherein in a center of the disk is an ultrasound (B-Mode) diagnostic transducer to verify target positions.
